(19) United States
(12) Patent Application Publication
(10) Pub. No.: US 2024/0127907 A1
(43) Pub. Date: Apr. 18, 2024

(54) BIOINFORMATICS PIPELINE AND ANNOTATION SYSTEMS FOR MICROBIAL GENETIC ANALYSIS

(71) Applicant: Contamination Source Identification LLC, Hershey, PA (US)

(72) Inventors: Colin Joseph Brislawn, Hershey, PA (US); Regina Lamendella, Hershey, PA (US); Vasilii Y. Tokarev, Hershey, PA (US); Justin Wright, Hershey, PA (US)

(21) Appl. No.: 18/547,753

(22) PCT Filed: Feb. 28, 2022
(86) PCT No.: PCT/US2022/018228

§ 371 Date: Aug. 24, 2023

Related U.S. Application Data
(60) Provisional application No. 63/154,646, filed on Feb. 26, 2021.

(51) Int. Cl. G16B 30/10; G16H 50/20; G16H 70/60

(52) U.S. Cl. CPC G16B 30/10; G16H 50/20; G16H 70/60

(57) ABSTRACT

A bioinformatics pipeline designed to analyze next generation sequence data as input and systematically quality filter, normalize, annotate, quantify, and identify microbial taxa of interest contained within microbial databases. In various embodiment, a bioinformatics pipeline may include a deep annotation strategy that confers an additional task that can be scaled to a limitless number of taxa of interest each time a taxa of interest is extracted for re-annotation.

### RELATED CASES

The present disclosure claims priority to U.S. provisional patent application, U.S. Application Ser. No. 62/154,646, filed Feb. 26, 2021, the entire disclosure of which is hereby incorporated by reference.

### TECHNICAL FIELD

The present disclosure is directed to a bioinformatics pipeline and deep annotation strategy designed to analyze next generation sequence data as input and systematically quality filter, normalize, annotate, quantify, and identify microbial taxa of interest contained within a series of microbial genomic databases.

### BACKGROUND

Detection and identification of microbial species is important for diagnosing and treating disease, and for identifying source contamination and preventing infection in various clinical, environmental or production contexts. Microbial species can include bacteria, viruses, protozoa, fungi, algae, amoebas, and slime molds.

Detection and identification of specific microbial species can be useful in the evaluation and treatment of patients and products, as well as facilities and environments, such as medical-related facilities like hospitals or clinics and food production environments like manufacturing plants or kitchens. Detection of specific microbial species is used, for example, in medical offices, in order to determine the presence and identity of pathogens causing infection to a patient, thus allowing for appropriate treatment and remedy. The same methods are used in healthcare, the food industry, and long-term care industry, hospitality industry, homeland security, aerospace and aviation, and even in the private sector.

Unfortunately, detection of microbial species from biological specimens obtained from such clinical, environmental or production contexts differ from detection of microbial species in controlled biological samples that are used in theoretical or research contexts. The raw biological specimens from such clinical, environmental or production contexts are not controlled or refined, and the time and expense that can be spent to obtain useable results from a biological sample in a theoretical or research context are significantly more than can be spent on obtaining results from a biological specimen in a clinical, environmental or production context.

Biological specimens are uniquely complex and can include biological materials that range from urine and feces to whole blood and serum to intact tissue. Biological specimens may comprise lipids, proteins, nuclear and mitochondrial DNA, RNA (i.e. tRNA, rRNA, mRNA), and will contain all of the above macromolecules for both the mammalian source of the specimen as well as any single-celled organisms (i.e. microbial species) that are present in the sample and infecting the host organism. Typically, biomarkers of a pathogen (e.g. DNA and RNA) are present at a significantly lower level than that of the source of the biological specimen, making isolation and detection difficult. Current techniques for microbial detection in the theoretical and research context rely on incubation and/or purification techniques applied to biological specimens in order to form relevant and actionable biological samples suitable for genetic analysis.

Incubation techniques can take up to 30 hours before the specimen can be analyzed thereby delaying the ability to act upon the results of detection of specific microbial species to achieve a favorable and timely outcome. Examples of such incubation techniques used to amplify genetic sequences are described, for example, in U.S. Pat. Nos. 8,313,931 (incubation for 20 hours) and 9,435,739 (incubation for 18-24 hours), and for the 3M™ Molecular Detection System (incubation for up to 30 hours as described in links available at https://multimedia.3m.com/mws/media/13533510/3m-molecular-detection-assay-2-1-monocytogenes-update.pdf and U.S. Publ. Appl. No. 2017/0219577 A1).

Purification techniques to isolate and detect biomarkers of microbial species by filtration, elution, or binding techniques as described, for example, in U.S. Pat. Nos. 9,062,303 and 8,383,340. Chaotropic agents such as DNase have been used to degrade proteins other than RNA in a clinical sample (Tan et al, J Biomed and Biotech, 2009, Turbo Dnase, available from ThermoFisher, U.S. Pat. No. 10,077,439); however, these methods do not allow for isolation of microbial RNA from non-microbial RNA. Similarly, methods to extract E. coli from human blood using co-purification with different lysis buffers have been disclosed (Brennecke et al, J Med Micro, 2017, 66: 301), but these methods rely on first extracting the target RNA from the biological specimen, and then degrading and removing any residual DNA. The use of specially prepared sterile plates to isolate specific strains of bacteria have been used in a research context to obtain relevant and actionable biological samples of each bacteria strain suitable for genetic analysis. See, material in links available at https://www.wrightlabs.org/metatranscriptomics_2, https://aac.asm.org/content/60/8/4722, https://www.nature.com/articles/s41598-018-21841-9. Unfortunately, these kinds of purification techniques require further processing and manipulation of the biological specimens, which may result in degradation of the genetic biomarkers of interest.

Various methods and systems for the detection of microbial DNA, particularly in the theoretical or research context are also known in the art. However, these techniques rely on detection of microbial DNA and cannot distinguish between the presence of live or dead microbial species. Moreover, because other kinds of DNA in a biological specimen typically overwhelm the relative amount of microbial DNA in that biological specimen, detection of microbial DNA using conventional DNA identification requires incubation or preparation of a biological sample in which sufficient numbers of the microbial species of interest are grown to reliably detect and identify those microbes.

Current bioinformatics pipeline processes also typically require more than two non-overlapping distinct reads to confirm a positive for viruses and a minimum RPM (read per million) compared to a standard expectation for bacteria fungi and parasites. One current bioinformatics pipeline, sequence-based ultrarapid pathogen identification (SURPI), incorporates a distinct annotation process and thresholding for viruses that requires two unique reads to cover distinct portions of the respective viral genome. (Naccache S N, Federman S, Veeraraghavan N, et al. A cloud-compatible bioinformatics pipeline for ultrarapid pathogen identification from next-generation sequencing of clinical samples. Genome Res. 2014; 24(7):1180-1192. doi:10.1101/gr.171934.113). The SURPI bioinformatics pipeline has been improved in various aspects in an updated pipeline known as SURPI+. (Miller S, Naccache S N, Samayoa E, et al. Laboratory validation of a clinical metagenomic sequencing assay for pathogen detection in cerebrospinal fluid. Genome Res. 2019; 29(5):831-842. doi:10.1101/gr.238170.118). Other examples of bioinformatics pipeline processing systems and techniques are described in PCT Appl. Nos. WO 2016/172643 A2, WO 2017/053446 A2 and WO 2020/038765 A1, U.S. Pat. Nos. 8,478,544 and 8,775,092, and Thomas, T., Gilbert, J. & Meyer, F. Metagenomics—a guide from sampling to data analysis. Microb Informatics Exp. 2, 3 (2012), https.://doi.org/10.1186/2042-5783-2-3.

Although techniques like SURPI and SURPI+ have improved genetic analysis of microbial species, there exists a need for a technique that can rapidly isolate and detect biomarkers of microbial species from biological specimens without significant, intentional, incubation or purification in order to facilitate more effective, efficient and rapid identification of microbial species in such biological specimens for the evaluation and treatment of patients and products, as well as facilities and environments.

### SUMMARY

In embodiments, a computer-implemented method for rapidly identifying pathogen sequence data from raw sequence data generated by a sequencing system can be executed on a processor. The processor can receive, from the sequencing system, raw sequence data sequenced from a sample that includes both human and pathogen genetic material. The processor can then preprocess the raw sequence data to filter out low quality sample sequence reads to generate a set of sample sequence reads. The processor can then extract, via an alignment technique, pathogen sequence data from the set of sample sequence reads to create reporting results of matches for a subset of the sample sequence reads of pathogen genetic material. The extracting process can include: comparing, via sequence alignment analysis, the set of sample sequence reads to host genomes representing known sequences of human genetic material stored in a host genome database to identify a human subset of the set of sample sequence that match a host genome sequence in the host genome database as background reads; removing the background reads from the set of sample sequence reads to create a pathogen dataset of sample sequence reads that is stored separate from the set of sample sequence reads; and comparing the pathogen dataset of sample sequence reads to a set of reference pathogen genomes representing known sequences of pathogen genetic material stored in a reference genome database to identify individual pathogens present in the pathogen dataset of sample sequence reads.

In embodiments, the sequence alignment analysis further includes performing, via a k-mer annotation methodology, an initial fast annotation of the pathogen dataset to identify a subset of pathogens at an unresolved taxonomic domain, phylum, class, order, family, or genus level and performing, via a sequence alignment methodology, a secondary slower annotation on the subset of pathogens at the unresolved taxonomic rank to identify pathogens at a species level. The reporting results are then displayed, communication and/or stored in a memory.

In embodiments, the raw sequence data sequenced from the sample can be bulk-filtered to enhance microbial RNA in the sample prior to sequencing by the sequencing system.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 shows a representative workflow diagram of one embodiment of a bulk filtration method.

FIG. 2 shows a representative chromatogram of a separation of microbial RNA.

FIG. 3 shows a representative chromatogram of a separation of microbial RNA.

FIG. 4 shows a representative block diagram of one embodiment of an overall workflow incorporating the pipeline processing method as part of an overall process for identifying microbial RNA in a biological specimen.

FIG. 5 shows a flow chart of a sequence-based ultrarapid pathogen identification method, according to an embodiment of the present disclosure.

FIG. 6 shows a flow chart of a bioinformatics pipeline, according to an embodiment of the present disclosure.

FIG. 7 shows a directed acyclic graph of a bioinformatics pipeline, according to an embodiment of the present disclosure.

FIG. 8 shows a directed acyclic graph of a bioinformatics pipeline, according to an embodiment of the present disclosure.

FIG. 9 shows a graph of an annotation assessment analysis for simulated Borrelia burgdorferi sequences.

FIG. 10 shows a graph of an annotation assessment analysis for Borrelia burgdorferi sequences recorded in a lab.

FIG. 11 shows a block diagram of a compute cluster, according to an embodiment of the present disclosure.

FIG. 12 shows a flow chart of a tracking overview process, according to an embodiment of the present disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

A description of a bulk filtering process used for more efficient and effective detection and identification of microbial species by using liquid chromatography is included in the attached Appendix A, which is incorporated by reference in its entirety.

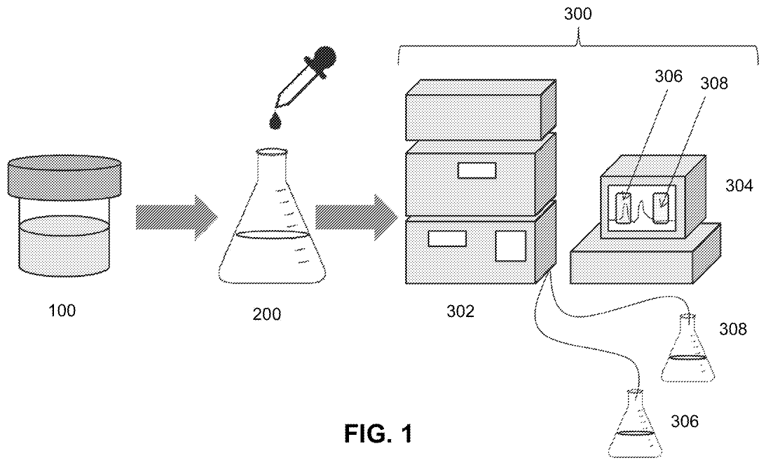

As depicted in FIG. 1, a clinical specimen 100 is obtained. In one embodiment, a clinical specimen is an environmental swab from a hard surface, wherein the swab is extracted in a buffered solution in a test vial. Buffered solutions may be comprised of 0.1× to 2.5× phosphate buffered saline (PBS), 0.05M to 1.5M Peptone water, or 30% to 50% guanidine hydrochloride with 0.1% to 1% maleic acid.

In another embodiment, a biological specimen is a tissue or excretion from a mammalian species, including but not limited to humans and domesticated animal species. The biological specimen includes, but is not limited to, whole blood, plasma, mucus, serum, urine, feces, cerebrospinal fluid, synovial fluid, and intact tissue. Clinical specimens may be obtained by blood draw, punch biopsy, stool culture, nasal swab, saliva sample, urinalysis, dermatology scraping, in addition to any other established protocol for collecting a specific form of biological specimen. Biological specimens contain large RNA molecules, small RNA molecules, tRNA molecules, rRNA molecules, mRNA molecules, denatured and non-denatured RNA molecules, microbial RNA molecules, non-microbial RNA molecules, genomic DNA molecules, protein molecules, and other macromolecules.

The biological specimen may be mechanically homogenized, cavitated by nitrogen, or sonicated, as appropriate using methods known in the art to prepare the specimen for the subsequent step of digesting in order to create a test sample. The biological specimen then undergoes the step of digesting 200. Digesting the clinical specimen may include enzymes, chaotropes, surfactants, detergents, and other additives known in the art. The process of digesting the biological specimen removes genomic DNA molecules, protein molecules and non-RNA macromolecules, and may occur at low temperatures to prevent degradation of the biological specimen and may optionally include additives known to preserve target molecules, as described in Table 1.

TABLE 1
Additives for preservation of target molecules while digesting clinical specimens.

| | | | Preferred |
|---|---|---|---|
| Inhibitor | Specificity | Use Level | Embodiment |
| PMSF | Cysteine and serine | 0.1 mM to 1 mM | Dissolve in ethanol |
| | proteases | | before using. |
| EDTA | Metalloproteases | 0.5 mM to 1.5 mM | Use when divalent |
| | | | cations are in the |
| | | | specimen. |
| Pepstatin A | Acid proteases | 1 μM to 2 μM | Prepare at 1 mg/mL |
| | | | and dilute |
| | | | immediately before |
| | | | use. |
| Leupeptin | Serine and thiol | 10 μM to 100 μM | Prepare 25 mg/mL |
| | proteases | | and dilute |
| | | | immediately before |
| | | | use. |
| Aprotinin | Serine proteases | 0.1 μM to 0.8 μM | Use 1 mg/mL of 5 mg/mL |
| | | | stock solution. |

In one embodiment, digesting the biological specimen begins by lysing the clinical specimen by interacting the biological specimen with Guainidinium thiocyanate, N-Lauroylsarcosine, and ethanol. In one embodiment, a buffer solution of 55% to 85% Guanidinium thiocyanate and 1% to 20% N-Lauroylsarcosine is mixed in equal volumes with a solution of 70% to 100% ethanol. In one embodiment, a buffer solution of 65% to 75% Guanidinium thiocyanate and 1% to 10% N-Lauroylsarcosine is mixed in equal volumes with a solution of 70% to 100% ethanol. In still another embodiment, a buffer solution of 65% to 75% Guanidinium thiocyanate and 1% to 10% N-Lauroylsarcosine is mixed in equal volumes with a solution of 90% to 100% ethanol.

In one embodiment, digesting the biological specimen by lysing the specimen continues by adding 1 to 3 volumes of a buffer solution and ethanol mixture to a volume of a clinical specimen. In one embodiment, 2 volumes of the buffer solution and ethanol mixture are added to a volume of a clinical specimen. In another embodiment, 200 μL to 400 μL of a buffer solution and ethanol mixture are added to a volume of a clinical specimen. In one embodiment, 250 μL to 350 μL of a buffer solution and ethanol mixture are added to a volume of a clinical specimen.

The aforementioned mixture with the biological specimen is mixed either through manual inversion, vortexing, or other means known in the art, then transferred through a silica or polypropylene filter by centrifugation at 500 g to 2000 g for 15 seconds to 90 seconds. In one embodiment, 15 seconds to 45 seconds of centrifugation are used. The material that passes through the filter is discarded, and the DNA and RNA remaining on the filter are subjected to further preparative steps.

Once the biological specimen is digested, the next step of preparing and washing the biological specimen may include interacting the clinical specimen with one of at least Guanidinium chloride, ethanol, 2-amino-2-(hydroxymethyl)-propane-1,3-dihydrochloride, and edetate disodium. In one embodiment, a mixture of 70% to 100% ethanol containing 25% to 55% Guanidinium chloride is added to the same silica or polypropylene filter at a volume of 300 μL to 500 μL and centrifuged at 500 g to 2000 g for 15 seconds to 90 seconds. The material that passes through the filter is discarded. In another embodiment, a mixture of 12 mg/m³ to 790 mg/m³ 2-amino-2-(hydroxymethyl) propane-1,3-dihydrochloride, and 20 mg/m³ to 2,000 mg/m³ edetate disodium, is prepared and added to the same silica or polypropylene filter at a volume of 400 μL to 900 μL and centrifuged at 500 g to 2000 g for 15 seconds to 60 seconds. The material that passes through the filter is discarded. In yet another embodiment, a second mixture of 12 mg/m³ to 790 mg/m³ 2-amino-2-(hydroxymethyl) propane-1,3-dihydrochloride, and 20 mg/m³ to 2,000 mg/m³ edetate disodium, is prepared and added to the same silica or polypropylene filter at a volume of 400 μL to 900 μL and centrifuged at 500 g to 2000 g for 30 seconds to 180 seconds. In another embodiment, water is added to the same silica or polypropylene filter and centrifuged at 50 g to 2000 g for 15 seconds to 60 seconds. The material that passes through the filter is the biological specimen and is retained.

In one embodiment, a mixture of 80% to 100% ethanol containing 30 to 47% Guanidinium chloride is added to the same silica or polypropylene filter and centrifuged at 500 g to 2000 g for 15 seconds to 60 seconds. In yet another embodiment, a mixture of 90% to 100% ethanol containing 35 to 45% Guanidinium chloride is added to the same silica or polypropylene filter and centrifuged at 500 g to 2000 g for 15 seconds to 45 seconds. The material that passes through the filter is the biological specimen and is retained.

In another embodiment, a mixture of 25 mg/m³ to 600 mg/m³ 2-amino-2-(hydroxymethyl) propane-1,3-dihydrochloride, and 50 mg/m³ to 1,000 mg/m³ edetate disodium, is prepared and added to the same silica or polypropylene filter at a volume of 200 μL to 500 μL and centrifuged at 500 g to 2000 g for 15 seconds to 60 seconds. In yet another embodiment, a mixture of 25 mg/m³ to 600 mg/m³ 2-amino-2-(hydroxymethyl) propane-1,3-dihydrochloride, and 50 mg/m³ to 1,000 mg/m³ edetate disodium, is prepared and added to the same silica or polypropylene filter at a volume of 200 μL to 500 μL and centrifuged at 500 g to 2000 g for 15 seconds to 60 seconds. In yet another embodiment, a mixture of 25 mg/m³ to 600 mg/m³ 2-amino-2-(hydroxymethyl) propane-1,3-dihydrochloride, and 50 mg/m³ to 1,000 mg/m³ edetate disodium, is prepared and added to the same silica or polypropylene filter at a volume of 200 μL to 500 μL and centrifuged at 500 g to 2000 g for 15 seconds to 45 seconds.

In another embodiment, a second mixture of 25 mg/m³ to 600 mg/m³ 2-amino-2-(hydroxymethyl) propane-1,3-dihydrochloride, and 50 mg/m³ to 1,000 mg/m³ edetate disodium, is prepared and added to the same silica or polypropylene filter at a volume of 400 μL to 900 μL and centrifuged at 500 g to 2000 g for 30 seconds to 180 seconds. In yet another embodiment, a second mixture of 25 mg/m³ to 600 mg/m³ 2-amino-2-(hydroxymethyl) propane-1,3-dihydrochloride, and 50 mg/m³ to 1,000 mg/m³ edetate disodium, is prepared and added to the same silica or polypropylene filter at a volume of 600 μL to 800 μL and centrifuged at 500 g to 2000 g for 30 seconds to 180 seconds. In yet another embodiment, a second mixture of 25 mg/m³ to 600 mg/m³ 2-amino-2-(hydroxymethyl) propane-1,3-dihydrochloride, and 50 mg/m³ to 1,000 mg/m³ edetate disodium, is prepared and added to the same silica or polypropylene filter at a volume of 600 μL to 800 μL and centrifuged at 500 g to 2000 g for 90 seconds to 150 seconds.

In another embodiment, water is added to the same silica or polypropylene filter and centrifuged at 50 g to 2000 g for 15 seconds to 45 seconds. In yet another embodiment, water is added to the same silica or polypropylene filter and centrifuged at 1,500 g to 2,000 g for 15 seconds to 45 seconds. In yet another embodiment, water is DNase/RNase-free water.

Digesting the biological specimen proceeds to the step of cleaning the biological specimen by interacting the biological specimen with one of at least Proteinase K, Guanidinium thiocyanate, N-Lauroylsarcosine, ethanol, 2-amino-2-(hydroxymethyl) propane-1,3-dihydrochloride, and edetate disodium. In one embodiment, 4U to 12U Proteinase K is added to the washed biological specimen and held at 45° C. to 65° C. for 15 minutes to 60 minutes. In one embodiment, 4U to 8U Proteinase K is added to the washed biological specimen and held at 45° C. to 65° C. for 15 minutes to 60 minutes. In yet another embodiment, 4U to 8U Proteinase K is added to the washed biological specimen and held at 50° C. to 60° C. for 15 minutes to 60 minutes. In yet another embodiment, 4U to 8U Proteinase K is added to the washed biological specimen and held at 50° C. to 60° C. for 20 minutes to 40 minutes. In another embodiment, for solid tissue or complex matrices, incubation proceeds for 1 to 3 hours. In another embodiment, 1 to 3 volumes of a mixture of 55% to 85% Guanidinium thiocyanate and 1% to 20% N-Lauroylsarcosine is added to the held biological specimen. In a preferred embodiment, 1 to 2 volumes of 55% to 85% Guanidinium thiocyanate and 1% to 20% N-Lauroylsarcosine is added to the held biological specimen. In yet another preferred embodiment, 1 to 2 volumes of 65% to 75% Guanidinium thiocyanate and 1% to 10% N-Lauroylsarcosine is added to the held biological specimen.

In one embodiment, a buffer solution of 55% to 85% Guanidinium thiocyanate and 1% to 20% N-Lauroylsarcosine is mixed in equal volumes with a solution of 70% to 100% ethanol. In one embodiment, a buffer solution of 65% to 75% Guanidinium thiocyanate and 1% to 10% N-Lauroylsarcosine is mixed in equal volumes with a solution of 70% to 100% ethanol. In still another embodiment, a buffer solution of 65% to 75% Guanidinium thiocyanate and 1% to 10% N-Lauroylsarcosine is mixed in equal volumes with a solution of 90% to 100% ethanol. In the embodiments, the biological specimen is now cleaned and ready to digest and isolate a test sample from the biological specimen.

Digesting the biological specimen proceeds to the step of isolating a test sample from the biological specimen by interacting the biological specimen with one of at least 55% to 85% Guanidinium thiocyanate, 1% to 20% N-Lauroylsarcosine, 70% to 100% ethanol, DNase I, 2-amino-2-(hydroxymethyl)-propane-1,3-dihydrochloride, and edetate disodium. In one embodiment, the biological specimen is first centrifuged at >10,000 g for 1 minute. In another embodiment, the supernatant of the centrifuged biological specimen is transferred to a new silica or polypropylene filter and centrifuged at >10,000 g for 1 minute. The material that passes through the filter is the biological specimen and is retained.

In another embodiment, 1 to 3 volumes of 70% to 100% ethanol are added to the biological specimen in a mixture of 25% to 85% Guanidinium thiocyanate and 1% to 20% N-Lauroylsarcosine; the resulting solution is mixed well, either through manual inversion or vortexing, or other methods known in the art. In a preferred embodiment, 1 to 2 volumes of 90% to 100% ethanol are added to the clinical specimen in a mixture of 65% to 76% Guanidinium thiocyanate and 1% to 10% N-Lauroylsarcosine, and the resulting solution is mixed well.

In another embodiment, the resulting solution is transferred to a silica or polypropylene filter and centrifuged at 10,000 g to 16,000 g for 15 to 60 seconds. In a preferred embodiment, the resulting solution is centrifuged for 15 seconds to 45 seconds. The material that passes through the filter is discarded.

In another embodiment, 200 μL to 600 μL of a mixture of 25% to 85% Guanidinium thiocyanate and 1% to 20% N-Lauroylsarcosine is added to the silica or polypropylene filter, and the filter is centrifuged for at 10,000 g to 16,000 g for 15 to 60 seconds. In a preferred embodiment, 65% to 75% Guanidinium thiocyanate and 1% to 10% N-Lauroylsarcosine is added to the silica or polypropylene filter, and the filter is centrifuged for at 10,000 g to 16,000 g for 15 to 45 seconds. The material that passes through the filter is discarded.

In another embodiment, 1U to 15U DNaseI is added to the silica or polypropylene filter and is held at room temperature for 10 minutes to 25 minutes. In a preferred embodiment, 3U to 8U DNaseI is added to the silica or polypropylene filter and is held at room temperature for 10 minutes to 25 minutes. In yet another preferred embodiment, 3U to 8U DNaseI is added to the silica or polypropylene filter and is held at room temperature for 12 minutes to 20 minutes. In another embodiment, 200 μL to 700 μL of a mixture of 25% to 55% Guanidinium chloride and 70% to 99% ethanol is added to the silica or polypropylene filter and centrifuged at 10,000 g to 16,000 g for 15 to 60 seconds. In a preferred embodiment, 300 μL to 500 μL of a mixture of 35% to 45% Guanidinium chloride and 95% to 99% ethanol is added to the silica or polypropylene filter and centrifuged at 10,000 g to 16,000 g for 15 to 45 seconds. In another embodiment, 400 □L to 900 μL of a mixture of 12 mg/m³ to 790 mg/m³ 2-amino-2-(hydroxymethyl) propane-1,3-dihydrochloride, and 20 mg/m³ to 2,000 mg/m³ edetate disodium is added to the silica or polypropylene filter and centrifuged at 10,000 g to 16,000 g for 15 to 60 seconds. In one embodiment, 600 μL to 800 μL of a mixture of 25 mg/m³ to 600 mg/m³ 2-amino-2-(hydroxymethyl) propane-1,3-dihydrochloride, and 50 mg/m³ to 1,000 mg/m³ edetate disodium is added to the silica or polypropylene filter and centrifuged at 10,000 g to 16,000 g for 15 to 45 seconds. In another embodiment, a second mixture of 200 μL to 700 μL of a mixture of 12 mg/m³ to 790 mg/m³ 2-amino-2-(hydroxymethyl) propane-1,3-dihydrochloride, and 20 mg/m³ to 2,000 mg/m³ edetate disodium is added to the silica or polypropylene filter and centrifuged at 10,000 g to 16,000 g for 60 to 180 seconds. In one embodiment, a second mixture of 300 μL to 500 μL of a mixture of 25 mg/m³ to 600 mg/m³ 2-amino-2-(hydroxymethyl) propane-1,3-dihydrochloride, and 50 mg/m³ to 1,000 mg/m³ edetate disodium is added to the silica or polypropylene filter and centrifuged at 10,000 g to 16,000 g for 90 to 150 seconds. In another embodiment, water is added to the same silica or polypropylene filter and centrifuged at 10,000 g to 16,000 g for 15 seconds to 450 seconds. The material that passes through the filter is the test sample and is retained.

The test sample from the biological specimen may optionally be further completed by boiling, treating with a denaturant, or storing at −70° C. In one embodiment, the test sample may be completed by boiling at 100° C. to 120° C. for 10 minutes to 30 minutes; in another embodiment, the test sample may be completed by boiling at 100° C. to 105° C. for 10 minutes to 20 minutes. In another embodiment, the test sample may be completed by treating the test sample with a denaturant with 25% to 50% of one of at least polysorbate 20; polysorbate 80; (1,1,3,3-Tetramethylbutyl)phenyl-poly ethylene glycol, Poly ethylene glycol tert-octylphenyl ether; and 4-(1,1,3,3-Tetramethylbutyl)phenyl-poly ethylene glycol, t Octylphenoxypolyethoxyethanol, Polyethylene glycol tert-octylphenyl ether.

In another embodiment, the test sample may be completed by storing at −70° C. The test sample contains large RNA molecules, small RNA molecules, tRNA molecules, rRNA molecules, mRNA molecules, denatured and non-denatured RNA molecules, microbial RNA molecules, non-microbial RNA molecules, and is now ready to undergo the step of using liquid chromatography 300 to isolate and collect microbial RNA molecules from the test sample.

Using liquid chromatography to bulk filter microbial RNA molecules from the test sample proceeds by injecting the test sample into a sample port of a liquid chromatography instrument 302. In one embodiment, injection volumes are 0.1 mL to 2 mL. In one embodiment, injection volumes are 0.5 to 1.5 mL. Within the liquid chromatography instrument 302, the test sample is carried by pumps at a prescribed flow rate over a porous stationary phase column using a liquid mixture referred to as a mobile phase. The process is controlled by a computer 304. The test sample is traditionally processed by the liquid chromatography instrument 302 to separate into components with similar properties, and the component of interest. Typically, all components of the test sample travel through the liquid chromatography instrument 302 to a waste line. However, in various embodiments of the present disclosure, either automatically or manually, regions with components of interest 306 are diverted to a collection container for further identification. In various embodiments, components of interest 306 in the form of microbial RNA are detected using either a diode array, UV-Vis, or a refractive index detector. Regions without components of interest 308 are detected as a flat line (“baseline”).

The liquid chromatography instrument filters the microbial RNA molecules from non-microbial RNA molecules to isolate and collect these molecules by decreasing and then increasing the amount of an organic buffer in a mobile phase in relation to an aqueous buffer in said mobile phase. In one embodiment, the amount of organic buffer in the mobile phase varies between 20% and 100%, as shown in Table 2.

TABLE 2
Mobile phase composition for isolation
and collection of microbial RNA.

| Time | % Aqueous Buffer | % Organic Buffer |
|---|---|---|
| 0 | 60-80 | 20-40 |
| 1 | 50-70 | 30-50 |
| 16 | 40-60 | 40-60 |
| 22 | 30-50 | 50-70 |
| 22.5 | 20-40 | 60-80 |
| 23 | 0-20 | 80-100 |
| 24 | 0-20 | 80-100 |
| 25 | 60-80 | 20-40 |
| 27 | 60-80 | 20-40 |

In one embodiment, amount of organic buffer in the mobile phase varies between 30% and 100%, as shown in Table 3.

TABLE 3
Mobile phase composition for isolation
and collection of microbial RNA.

| Time | % Aqueous Buffer | % Organic Buffer |
|---|---|---|
| 0 | 60-70 | 30-40 |
| 1 | 55-65 | 35-45 |
| 16 | 35-45 | 55-65 |
| 22 | 30-40 | 60-70 |
| 22.5 | 25-35 | 65-75 |
| 23 | 0-10 | 90-100 |
| 24 | 0-10 | 90-100 |
| 25 | 60-70 | 30-40 |
| 27 | 60-70 | 30-40 |

In some aspects, the void volume elutes in the liquid chromatography mobile phase when the percent aqueous buffer is greater than about 40%, in some aspects greater than about 45%, in some aspects greater than about 50%, in some aspects greater than about 55%, and in some aspects greater than about 60%. In some aspects, the void volume elutes in the liquid chromatography mobile phase when the percent organic buffer is less than about 60%, in some aspects less than about 55%, in some aspects less than about 50%, in some aspects less than about 45%, and in some aspects less than about 40%.

In one embodiment, the flow rate of the liquid chromatography mobile phase, as delivered by the pumps is 0.5 mL to 3.5 mL/min. In one embodiment, the flow rate of the liquid chromatography mobile phase, as delivered by the pumps, is 1 mL/min to 2.5 mL/min. In yet another embodiment, the flow rate of the liquid chromatography mobile phase, as delivered by the pumps, is 1 mL/min to 1.5 mL/min.

In some aspects, a flow rate of the mobile phase in the liquid is about 0.5 mL/min. to about 3.5 mL/min., in some aspects about 550 μL/min. to about 2 mL/min., in some aspects about 600 μL/min. to about 1 mL/min., and in some other aspects about 650 μL/min. to about 850 μL/min.

The mobile phase is delivered as a mixture of two buffers, on an organic buffer and the other an aqueous buffer. In one embodiment, the aqueous buffer is comprised of at least one of at least 0.05 to 0.9M triethylammonium acetate, phosphoric acid, citric acid, ammonium bicarbonate, formic acid, lactic acid, 2-[4-(2-hydroxyethyl)piperazin-1-yl]ethanesulfonic acid, maleic acid, diethanolamine, piperidine, ethanolamine, and triethanolamine. In particular, a 0.05M to 0.5M buffer comprised of at least one of triethylammonium acetate, formic acid, lactic acid, 2-[4-(2-hydroxyethyl)piperazin-1-yl]ethanesulfonic acid, maleic acid, triethanolamine, and piperidine is used; more preferably a 0.05 M to 0.2M solution of aqueous buffer of at least one of triethylammonium acetate, formic acid, 2-[4-(2-hydroxyethyl)piperazin-1-yl]ethanesulfonic acid, maleic acid, and triethanolamine, is used.

In another embodiment, the organic buffer is comprised of a mixture of a 0.05 M to 0.9 M aqueous buffer solution in one of at least 5% to 60% acetonitrile, methanol, ethanol, 1-propanol, 2-propanol, acetone, and tetrahydrofuran; in particular, the organic buffer is comprised of a mixture of 0.05 M to 0.5 M aqueous buffer solution in at least one of 7% to 50% acetonitrile, methanol, ethanol, 1-propanol, and acetone. In one embodiment, the organic buffer is comprised of a mixture of 0.05 M to 0.5 M aqueous buffer solution in at least one of 10% to 40% acetonitrile, methanol, and acetone.

In the method according to various embodiments, a non-polar compound serves as the porous stationary phase column, either in the form of polymeric beads, polymeric microspheres, or a polymerized block. Irrespective of its precise form, the polymeric stationary phase column is porous in nature, which means that the polymeric stationary phase column is characterized by pores. The stationary phase column material may be commercially available and be uncoated or coated with specialized polymeric compounds designed to cover pores on the bead or microsphere surface to prevent microbial RNA from irreversibly interacting with the stationary phase column material. Within an outer structure of the stationary phase column are polymeric microspheres, preferably comprised of alkylated non-porous polystyrene-divinylbenzene copolymer. The stationary phase column is provided with a microsphere particle size of 8.0 lam to 50 lam, preferably 8.0 lam to 25 lam. The resulting pore size of the stationary phase column is 1000 Å to 5000 Å, in particular a pore size of 1000 Å to 4000 Å, more preferably 2000 Å to 4000 Å, or 2500 to 4000 Å. The stationary phase column may have dimensions of 4 mm to 30 mm wide by 40 mm to 150 mm long, preferably 5 mm to 10 mm wide by 40 mm to 100 mm long, even more preferably 7 mm to 9 mm wide by 40 to 60 mm long. According to one embodiment, the stationary phase column may be operated at ambient temperature, or more preferably controlled at a temperature of 20° C. to 27° C.

Microbial RNA is filtered and isolated from non-microbial RNA in the stationary phase column through selective interactions with the mobile phase and stationary phase column microspheres and pores. As the filtration and isolation occurs, the microbial and non-microbial RNA exit, or elute, from the stationary phase column at different times, and are detected by a detector, with microbial RNA eluting in the void volume of the column. In one embodiment, detection of the isolated microbial RNA is accomplished with a UV-Vis or diode array detector, coupled to the liquid chromatography instrument at the exit of the stationary phase column, at 200 nm to 220 nm, in particular at 203 nm to 217 nm, and still more preferably at 205 nm to 215 nm. In another embodiment, detection of the isolated microbial RNA is accomplished with a UV-Vis or diode array detector, coupled to the liquid chromatography instrument at the exit of the stationary phase column, at 250 nm to 270 nm, in particular 257 nm to 267 nm, and still more preferably 255 nm to 265 nm. In another embodiment, detection of the isolated microbial RNA is accomplished with a refractive index detector, coupled to the liquid chromatography instrument at the exit of the stationary phase column, set with a refractive index default range of 0.75 RI to 2.00 RI, in particular a default range of 0.9 RI to 1.90 RI, and still more preferably a default range of 1.00 RI to 1.75 RI.

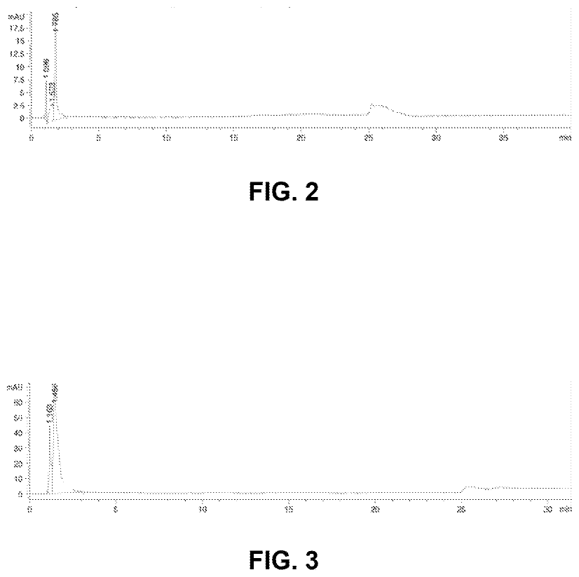

Isolated microbial RNA is detected in a trace of data referred to as a chromatogram. Exemplary chromatograms of isolated microbial RNA are found in FIGS. 2 and 3. The peaks observed in the window of 0 minutes to 3 minutes, correspond to isolated microbial RNA. Non-microbial RNA would be detected in the window of 7 minutes to 15 minutes, as disclosed by Ketterer et al (c.f U.S. Pat. No. 8,383,340). When the microbial RNA is detected, the mobile phase, containing compounds of interest (e.g. microbial RNA), is diverted from the waste line to a sample collection vial for future identification or experiments.

In some aspects, the sample collection of mobile phase from liquid chromatography includes the void volume and at least a portion of the mobile phase corresponding to the peaks relating to non-microbial RNA. In some aspects, the mobile phase containing compounds of interest (e.g., microbial RNA) may be collected in one or more fractions of eluted sample. In some aspects, a plurality of fractions of eluted sample are collected based upon time, volume, or both, as the mobile phase containing any compounds of interest elute from the column.

In some aspects, each fraction is collected from the column at a period of time between about 5 seconds and about 1 minute, in some aspects between about 10 seconds and about 45 seconds, and in some aspects between about 15 seconds and about 30 seconds. In some aspects, each fraction collected has a volume between about 100 μL to about 1 mL, in some aspects between about 125 μL to about 750 μL, in some aspects between about 150 μL to about 500 μL, and in some aspects between about 175 μL to about 250 μL.

In some aspects, at least a portion of the void volume may be collected in a quantity of desired fractions between at least one 1 fraction and up to about 72 fractions, in some aspects at least 1 fraction up to about 36 fractions, and in some other aspect at least 1 fraction up to about 24 fractions.

After the sample volume is collected from the liquid chromatography in one or more fractions, each of the one or more fractions may be subjected to gene sequencing.

In one embodiment, microbial RNA is detected from one or more fractions eluted from the void volume using liquid chromatography, wherein the microbial RNA is detected from at least one of the one or more fractions eluted from the void volume using gene sequencing.

In some aspects, each fraction is subjected to dehydration prior to gene sequencing, wherein each fraction is dehydrated to a volume between about 15 μL and about 500 μL, in some aspects between about 20 μL and about 100 μL, in some aspects between about 25 μL and about 75 μL, and in some preferred aspects between about 35 μL and about 65 μL.

In some aspects, a control may be introduced into the biological sample to normalize and/or monitor the microbial RNA relative to the non-microbial RNA. In some aspects, the control may be introduced into the biological sample prior to the step of digesting the biological specimen, after the biological specimen is digested, with the test sample that is introduced into the liquid chromatography, or when the desired sample is subjected to gene sequencing. The control is preferably chosen such as to elute from the column within both the void volume and the normal sample separate volume. The control may be chosen from any desired source that does not interfere with the microbial RNA or the sample RNA. In some preferred aspects, the control is a synthetically derived RNA such as an ERCC RNA control, such as ERCC RNA control Ambion™ commercially available from ThermoFisher Scientific.

Using various embodiments in accordance with this disclosure, it is possible to simultaneously isolate and collect all microbial RNA of interest, thereby allowing for future identification of all viable (live) microbial species within a biological specimen, or subsequent structural elucidation, quantitation, or qualitative analysis of the microbial RNA. Various embodiments of the present disclosure allow for simultaneous isolation and collection of microbial RNA from gram positive bacteria, gram negative bacteria, bacterial spores, enveloped viruses, non-enveloped viruses, RNA viruses, fungi, yeast, and protozoa. Exemplary microbial species within the test sample that are filtered, isolated and collected as microbial RNA using the method of the present disclosure, are found in Table 4.

TABLE 4
Microbial species detected by isolating and
collecting RNA from clinical specimens.

| | Gram | Gram | Virus, Protozoa, |
|---|---|---|---|
| Species Detected | (+) | (−) | Yeast, or Fungi |
| E. coli | | Y | |
| E. coli O157 | | Y | |
| S. aureus | Y | | |
| Campylobacter jejuni | | Y | |
| S. typhi | | Y | |
| C. perfringens | Y | | |
| C. botulinum | Y | | |
| B. burgdorferi | | Y | |
| Norovirus | | | Y |
| B. mayonii | | Y | |
| C. parvum | | | Y |
| L. monocytogenes | Y | | |
| S. sonnei | | | |
| L. pneumophila | | Y | |
| P. aeruginosa | | Y | |
| C. albicans | | | Y |
| M. tuberculosis | Y | | |

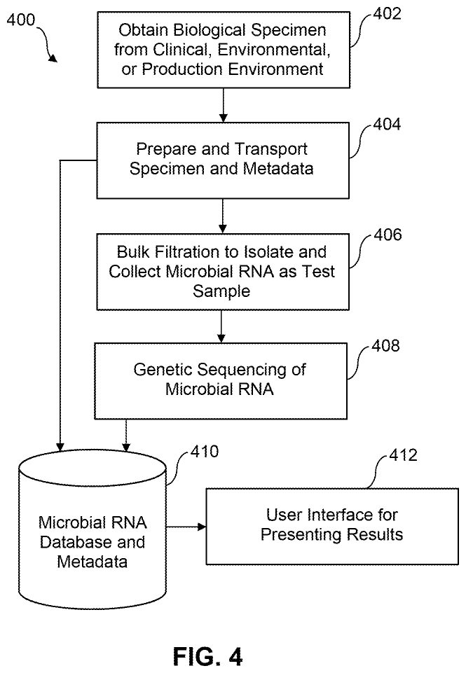

FIG. 4 shows a representative block diagram of one embodiment of an overall workflow 400 incorporating the pipeline method as part of an overall process for identifying microbial RNA in a biological specimen. At 402, a biological specimen is sampled and collected from either a clinical, production or environmental context. Although the biological specimen may be processed in accordance with various embodiments which equipment located proximate the context where it was sampled, in other embodiments, the biological specimen is transported at 404 to a different facility to perform the filtration method in accordance with various embodiments. Through the various embodiments, the biological specimen is transformed into a test sample to allow for isolation and collection of microbial RNA using liquid chromatography. At 404, a test sample containing the isolated and collected microbial RNA is produced in a void volume of an HPLC, in accordance with the various embodiments. The test sample with only the microbial RNA is then sequenced at 408 to identify all viable (live) microbial species within the test sample. In other embodiments, 408 can include structural elucidation, quantitation, or qualitative analysis of the microbial RNA. The results of the identification of viable microbial species from the biological specimen can be collected in a database 410 and presented or reported out via a user interface 412, accessible via a secure network interface.

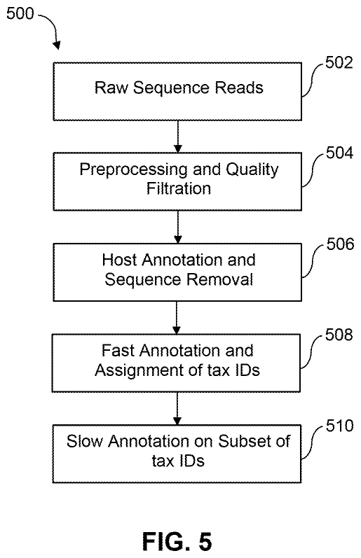

Referring to FIG. 5, a flowchart of a bioinformatics pipeline process 500 is depicted, according to an embodiment. The bioinformatics pipeline process 500 is designed to analyze next generation sequence data (FASTQ format) as input and systematically quality filter, normalize, annotate, quantify, and identify microbial taxa of interest contained within a microbial polished database above validated thresholds. A microbial polished database is one which improves the quality of pathogenic and nonpathogenic genomes within the database by selectively isolating regions of the genomes that are contaminated. In embodiments, the database stores genomes within the database as FASTQ formatted data.

At raw sequence reads step 502, raw sequence data sequenced from a sample that includes both human and pathogen genetic material is received from a sequencing system. In an embodiment, an Illumina sequencer is used. In most embodiments real sequence data can be used. In some embodiments simulated sequence data or a hybrid of simulated and real sequence data can be used.

At preprocessing and quality filtration step 504, low quality sample sequence reads are filtered out from the raw sequence data to generate a set of sample sequence reads. For example, the quality threshold can be based on a Q score of a moving window average, with the moving window size and Q score may be set at 4 bp:20Q, 75 bp min length. In embodiments, a range of 3 to 10 base pairs may be used to determine the window size for which a quality score is calculated. In embodiments, a quality threshold within the range of 4:15 to 4:30 is used. In one embodiment, a minimum length requirement within the range of 50 bp to 150 bp is used. In one embodiment, a minimum length requirement within the range of 60 bp to 120 bp is used. In one embodiment, a minimum length requirement of 75 bp is used.

At host annotation and sequence removal step 506, an alignment technique is used to extract pathogen sequence data from the set of sample sequence reads to create reporting results of matches for a subset of the sample sequence reads of pathogen genetic material. This extraction process includes comparing the set of sample sequence reads to host genomes representing known sequences of human genetic material stored in a host genome database to identify a human subset of the set of sample sequence that match a host genome sequence in the host genome database as background reads. The background reads are then removed from the set of sample sequence reads to create a pathogen dataset of sample sequence reads that is stored separately from the set of sample sequence reads.

At fast annotation step 508, a fast annotation, such as a k-mer annotation, is used to assign all taxa a tax ID. In embodiments, the tax IDs are derived from a data dictionary of the National Center for Biotechnology (NCBI) tax IDs organized in a structured hierarchy based on their associated phylogenetic relationships. Upon the completion of the fast k-mer annotation, each annotated sequence is assigned a tax ID which in turn is queried and collated during slow annotation step 510.

For certain microbes, k-mer annotation methodology can annotate and resolve experimental sequences up to the genus taxonomic levels more quickly, but it can be difficult to get species level identifications as the speed of the match comes at the expense of specificity. A fast annotation allows rapid, preliminary identification of taxonomic levels which can then be used to isolate a subset of taxa for further slow annotation, reducing the time required to achieve results without sacrificing detail or accuracy.

In embodiments, fast annotation step 508 can comprise a k-mer annotation of a polished or preselected database. In such embodiments, the prior organization of the database can remedy some specificity flaws that conventionally prevent k-mer annotation from being as effective as other annotation strategies, such as SNAP alignment, when run on unpolished genomic databases. Particularly, using k-mer annotation on a polished database can reduce the number of false-positives without affecting the accuracy of true positives.

At slow annotation step 510, a deep annotation strategy is used over a subset of tax ID's that are of interest. Selecting a subset of tax IDs for slow annotation allows for quicker turnaround than running a slow annotation on all tax IDs.

In embodiments, taxa of interest can be prechosen based on project information, source location, or other information provided by a user. In embodiments, a user can select desired taxa of interest from a list or menu provided in a user interface. In some embodiments, a user can select a specific infection or other condition of interest and taxa associated with the condition will automatically be added to the tax ID subset for slow annotation step 510.

In embodiments, databases used during alignment can be selected by a user. Database selection can be based on taxa of interest, conditions of interest, or other user provided information. In embodiments, an appropriate genomic database may be automatically chosen. Selecting an alignment database that is closely tailored to the taxa of interest for the slow annotation can further improve the speed of the annotation.

In embodiments, the bioinformatics pipeline is constructed and organized to permit scalable and reproducible analyses. In one embodiment, the bioinformatics pipeline is organized as a snakemake workflow. In one embodiment, a bioinformatics pipeline management tool such as nextflow or Common Workflow Language (CWL) is used to organize the bioinformatics pipeline. The bioinformatics pipeline is executed within a computational cluster using a single command written as a wrapper. The bioinformatics pipeline can be executed with a single command through a customized Lab Information Management System (LIMS) platform for the Enterprise Science Platform (ESP), which provides an easy to use graphical user interface (GUI) for informatically inexperienced users to easily run the bioinformatics pipeline in an audit tracked compliant system. Execution of the bioinformatics pipeline is CLIA compliant. Further, the LIMS platform has improved accession of files and barcodes are transposed into file names for clear reference.

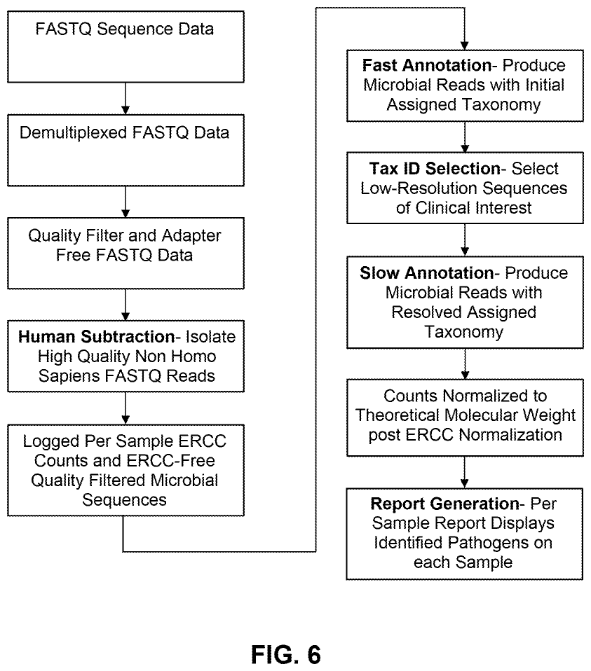

Referring to FIG. 6, a bioinformatics pipeline, according to an embodiment, is intentionally designed to execute all desired informatics processes in a systematic and sequential order. To begin, quality filtration is conducted by removal of poor quality reads. For example, the quality threshold can be set at 4:20, 75 bp min length. In embodiments, a range of 3 to 10 base pairs may be used to determine the window size for which a quality score is calculated. In embodiments, a quality threshold within the range of 4:15 to 4:30 is used. In one embodiment, a minimum length requirement within the range of 50 bp to 150 bp is used. In one embodiment, a minimum length requirement within the range of 60 bp to 120 bp is used. In one embodiment, a minimum length requirement of 75 bp is used. In an embodiment, fastp, an ultra-fast all-in-one FASTQ preprocessor, is used to perform quality filtering. In other embodiments, other FASTQ processors, such as Trimmomatic, bbduk, or fastx can be used.

Then host (e.g., Homo sapiens) annotation and sequence removal is conducted within a threshold range of 0.1-1.0 per sequence k-mer fraction using an annotation strategy like KRAKEN2. In embodiments, a different annotation strategy such as KRAKEN-Uniq, CLARK, or BBMAP could be used. In one embodiment, host annotation and sequence removal is conducted within a threshold range of 0.2 to 0.8 per sequence k-mer fraction. In one embodiment, host annotation and sequence removal is conducted within a threshold range of 0.4 to 0.6 per sequence k-mer fraction. In one embodiment, host annotation and sequence removal is conducted within a 0.4 per sequence k-mer fraction.

Once the host annotation and sequence have been removed an External RNA Controls Consortium (ERCC) control sequence annotation and sequence removal is conducted. In one embodiment, minimap2 is used as a pairwise aligner for ERCC filtration. In other embodiments, emboss, needle, GMAP, or other ERCC filtration processes can be used. Next, initial microbial annotation (fast annotation) is conducted with a threshold of 0.6 per sequence k-mer fraction. In one embodiment, initial microbial annotation and sequence removal is conducted within a threshold range of 0.2 to 0.8 per sequence k-mer fraction. In one embodiment, initial microbial annotation and sequence removal is conducted within a threshold range of 0.4 to 0.6 per sequence k-mer fraction. In one embodiment, initial microbial annotation and sequence removal is conducted within a 0.4 per sequence k-mer fraction.

In some embodiments, the threshold for host annotation is less than the threshold microbial annotation. In some embodiments, the thresholds are the same. In some embodiments, the threshold for host annotation is greater than the threshold for microbial annotation. Generally, a lower threshold for removing host annotation and sequence removal is used to efficiently remove host reads before upping the stringency of the initial microbial annotation to ensure quality annotations.

Following annotation and sequence removal, a select slower annotation of select microbes is conducted, including selecting genera of interest, collating associated sequences, and annotating select sequences against a comprehensive polished genome database using BLAST alignment. In embodiments, other local sequence alignments can be used, including parasail, GOTTCHA, or BWA-Mem. This annotation typically has a threshold of a minimum 0.001 e-value and a minimum of 95% of top hits must align with same species for a call to be made. In one embodiment, an e value of 0.0001 to 1×10⁻⁶ is used. In one embodiment, an e value of 0.0001 to 1×10⁻⁴ is used. In one embodiment, an e value of 0.0001 to 1×10⁻³ is used. In one embodiment, the percent hits of that align with same species for a call to be made is within 55% to 100%. In one embodiment, the percent hits of that align with same species for a call to be made is within 75% to 100%. In one embodiment, the percent hits of that align with same species for a call to be made is within 90% to 100%. In one embodiment, the percent of hits that align with same species for a call to be made is 100%.

All computational nodes on the compute cluster are streamlined, scaled, and optimized. The bioinformatics pipeline scales processing across entire runs, the “input” for each command is an entire run folder. Therefore, the process does not need to be repeated for each individual sample on each run.

The bioinformatics pipeline can preload databases into RAM during processing to save greatly on computational time and sample processing can be scaled so that samples can continue processing further downstream the pipeline even if additional samples on the run are still further behind in the pipeline chain. Further, all results are collated in a final table at the end of processing of all samples on a given run. The removal of sequential “loop processes” optimizes processing time by removing computational “down-time” in which samples are “waiting” for remaining files to complete. These optimizations when paired with the bulk filtration process significantly reduce the time and computational resources necessary to identify microbial taxa of interest.

Strategic database preloading into RAM can allow optimization of the workflow. In one embodiment, the workflow is a snakemake workflow. Database preloading can be particularly effective after filtration is complete and before annotation begins.

In embodiments, the bioinformatics pipeline first uses fast annotation with k-mer annotation and then uses a secondary, follow-up global or local alignment annotation process that is computationally slower in comparison to the primary fast k-mer annotation. A bioinformatics pipeline according to an embodiment of the present disclosure was constructed and validated with single-ended 150 base pair reads. For certain microbes, k-mer annotation can annotate and resolve experimental sequences up to the genus taxonomic levels more quickly, but it can be difficult to get species level identifications as the speed of the match comes at the expense of specificity.

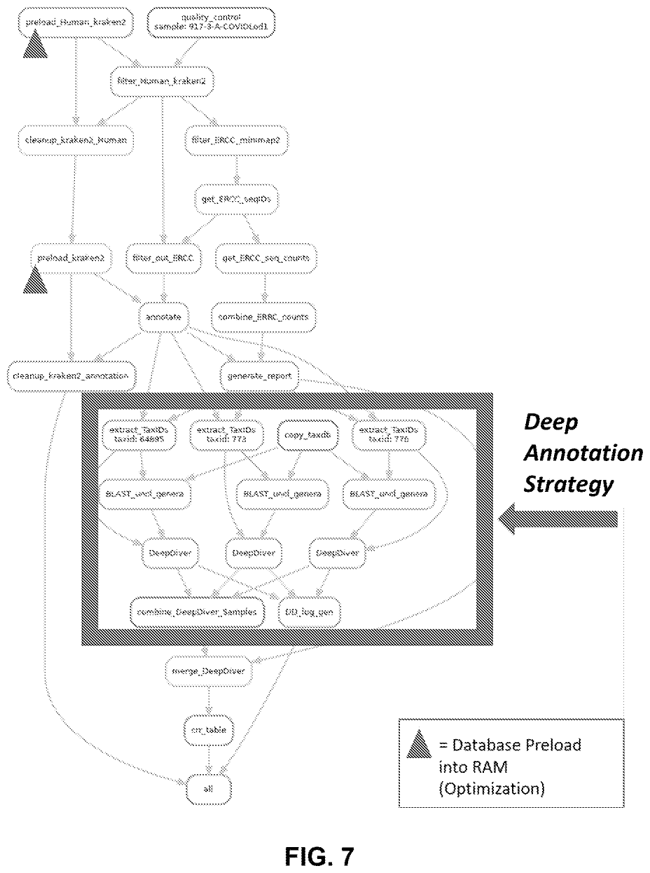

Referring to FIG. 7, a directed acyclic graph (DAG) plot of reannotation of all Coxiella & Bartonella genera annotated reads undergoing deep annotation strategy re-annotation is depicted. The DAG plot provides a comprehensive overview of all sequential tasks carried out on each individual FASTQ (sequence file) using the bioinformatics pipeline of the present disclosure. This includes database preload steps that permit accelerated processing on the computational cluster. Additionally, the DAG plot showcases the flexibility/scalability of the deep annotation strategy, and how each extracted tax ID of interest for re-annotation confers an additional task/job that can be scaled to a limitless number of taxa of interest.

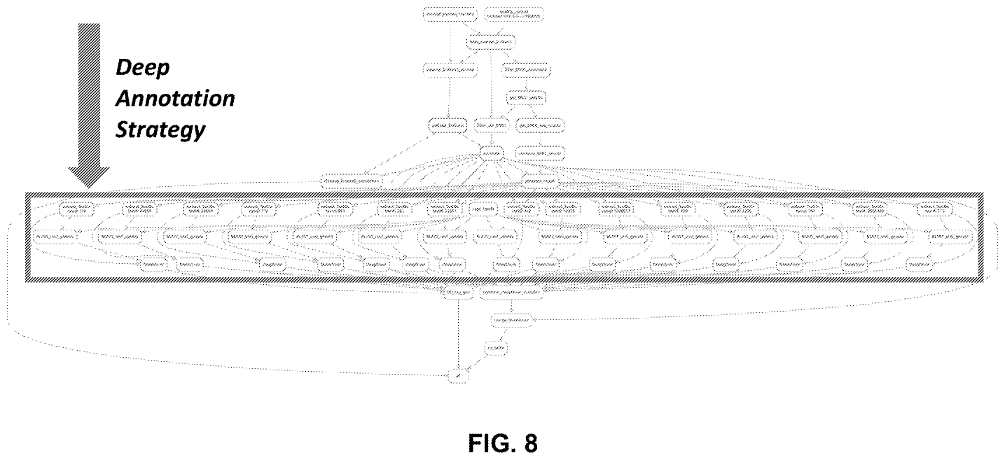

Referring to FIG. 8, reannotation of all genera annotated reads that are associated with tick-borne disease are undergoing deep annotation strategy re-annotation. Deep annotation strategy permits flexibility for the user, allowing for selection of microbial genera of interest to refine/re-annotate.

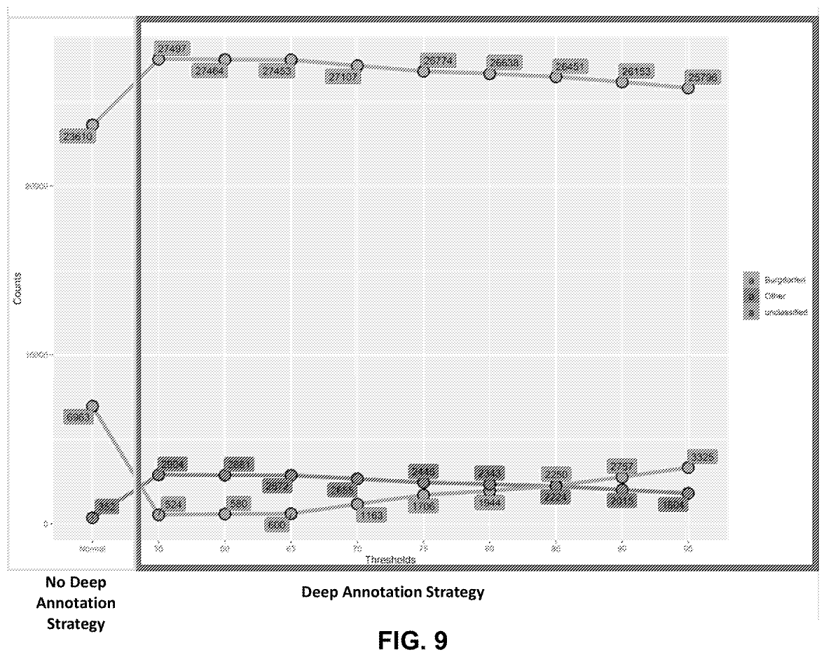

An in silico validation experiment was conducted in which 35000 simulated Borrelia burgdorferi sequences were generated using a sequence read simulation tool. After filtration of the simulated sequences, a total of 30,925 sequences remained for annotation assessment analysis. The remaining sequences were used to investigate the ability of deep annotation strategy, and particularly follow up annotation, to better resolve and annotate sequences with a known genomic origin or outcome. In this experiment Borrelia burgdorferi was used as it is the bacterial causal agent of Lyme Disease. The results of this experiment are depicted in FIG. 9 as well as Table 5 below:

TABLE 5
Annotation Assessment of Simulated Borrelia burgdorferi Sequences

| | No | | | | | | | | | |
|---|---|---|---|---|---|---|---|---|---|---|
| | Deep | 55% | 60% | 65% | 70% | 75% | 80% | 85% | 90% | 95% |
| | Diver | DD | DD | DD | DD | DD | DD | DD | DD | DD |
| Annotated as | 23610 | 27497 | 27464 | 27107 | 26774 | 26638 | 26451 | 26638 | 26153 | 25796 |
| Borrella | | | | | | | | | | |
| burgdoreri | | | | | | | | | | |
| Annotated as | 6963 | 524 | 580 | 600 | 1163 | 1706 | 1944 | 2250 | 2757 | 3325 |
| Borrella | | | | | | | | | | |
| unclassified | | | | | | | | | | |
| Annotated as | 352 | 2904 | 2881 | 2872 | 2655 | 2445 | 2343 | 2224 | 2015 | 1804 |
| Other | | | | | | | | | | |
| Total | 30925 | | | | | | | | | |
| TPR % | 76.35 | 88.9 | 88.8 | 88.8 | 88.7 | 86.6 | 86.1 | 85.5 | 84.6 | 83.4 |
| FPR % | 1.49 | 10.6 | 10.5 | 10.5 | 9.8 | 9.1 | 8.8 | 8.4 | 7.7 | 7.0 |
| Sensitivity % | 77.23 | 98.1 | 97.9 | 97.9 | 95.9 | 94.0 | 93.2 | 92.1 | 90.5 | 88.6 |
| (TP/(TP + FN)) | | | | | | | | | | |
| FDR % | 1.47 | 9.5 | 9.5 | 9.5 | 8.9 | 8.4 | 8.1 | 7.8 | 7.1 | 6.5 |
| (FP/(TP + FP)) | | | | | | | | | | |

The deep annotation threshold was parameter swept to identify the optimal thresholding for pathogen calling. In FIG. 9, the percent of genomic genomes aligned against to permit taxa assignment was stretched from 55% of alignments to 95%. It is clearly observed that deep annotation strategy substantially increases sensitivity from 77% to a range of 88.58%-98.13%. The consequence of deep annotation strategy is a higher False Discovery Rate (FDR) or species missannotation, in particular in the lower threshold range. In one embodiment, the percent of genomic genomes aligned against to permit taxa assignment is stretched from 55% to 100%. In one embodiment, the percent of genomic genomes aligned against to permit taxa assignment is stretched from 75% to 100%. In one embodiment, the percent of genomic genomes aligned against to permit taxa assignment is stretched from 90% to 100%.

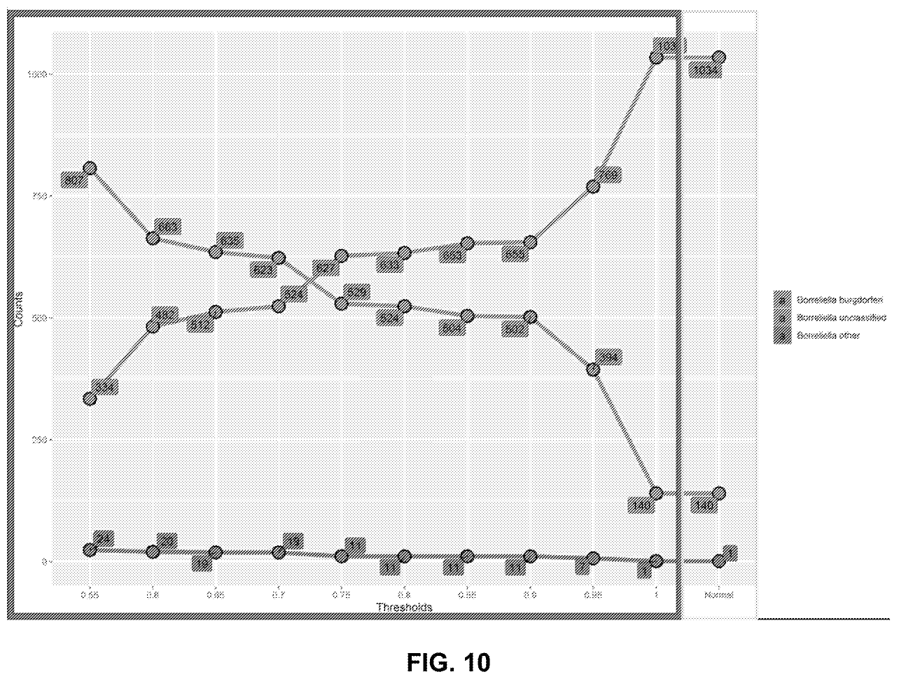

Referring to FIG. 10 and Table 6 below, a similar validation experiment was conducted;

however the experiment used real-world data generated in a lab. For the experiment, a controlled amount of Borrelia burgdorferi cells were spiked into control urine (Lyme disease negative). In this real world scenario, an increased ability for the deep annotation strategy is observed to better resolve target annotations, and the FDR remains relatively low in comparison to single step annotation strategies.

TABLE 6
Annotation Assessment of Borrelia burgdorferi Sequences from Lab Testing

| | No | | | | | | | | | |
|---|---|---|---|---|---|---|---|---|---|---|
| | Deep | 55% | 60% | 65% | 70% | 75% | 80% | 85% | 90% | 95% |
| | Diver | DD | DD | DD | DD | DD | DD | DD | DD | DD |
| Annotated as | 140 | 807 | 663 | 635 | 623 | 529 | 524 | 504 | 502 | 394 |
| Borrella | | | | | | | | | | |
| burgdoreri | | | | | | | | | | |
| Annotated as | 1034 | 334 | 482 | 512 | 524 | 627 | 633 | 653 | 655 | 769 |
| Borrella | | | | | | | | | | |
| unclassified | | | | | | | | | | |
| Annotated as | 1175 | 24 | 20 | 19 | 19 | 11 | 11 | 11 | 11 | 7 |
| Other | | | | | | | | | | |
| Total | 1175 | | | | | | | | | |
| TPR % | 11.91 | 69.3 | 56.9 | 54.5 | 53.4 | 45.3 | 44.9 | 43.1 | 43.0 | 33.7 |
| FPR % | 0.09 | 2.1 | 1.7 | 1.6 | 1.6 | 0.9 | 0.9 | 0.9 | 0.9 | 0.6 |
| Sensitivity % | 11.93 | 70.7 | 57..9 | 55.4 | 54.3 | 45.7 | 45.3 | 43.6 | 43.4 | 33.9 |
| (TP/(TP + FN)) | | | | | | | | | | |
| FDR % | 0.71 | 2.9 | 2.9 | 3.0 | 2.0 | 2.0 | 2.1 | 2.1 | 2.1 | 1.7 |
| (FP/(TP + FP)) | | | | | | | | | | |

In embodiments, the deep annotation strategy utilizes a data dictionary of NCBI tax ids organized in a structured hierarchy based on their associated phylogenetic relationships. Upon the completion of the fast k-mer annotation, each annotated sequence is assigned a tax id which in turn is queried and collated during the deep annotation stage of the bioinformatics pipeline. The deep annotation strategy can be flexible in that user can select any available microbial tax id of interest for subsequent speciation via re-annotation. The focus is on species to provide earlier detection and diagnosis for directed antibiotic treatment. To achieve this end, the deep annotation strategy increases taxa resolution at the species level to dramatically increase sensitivity of results.

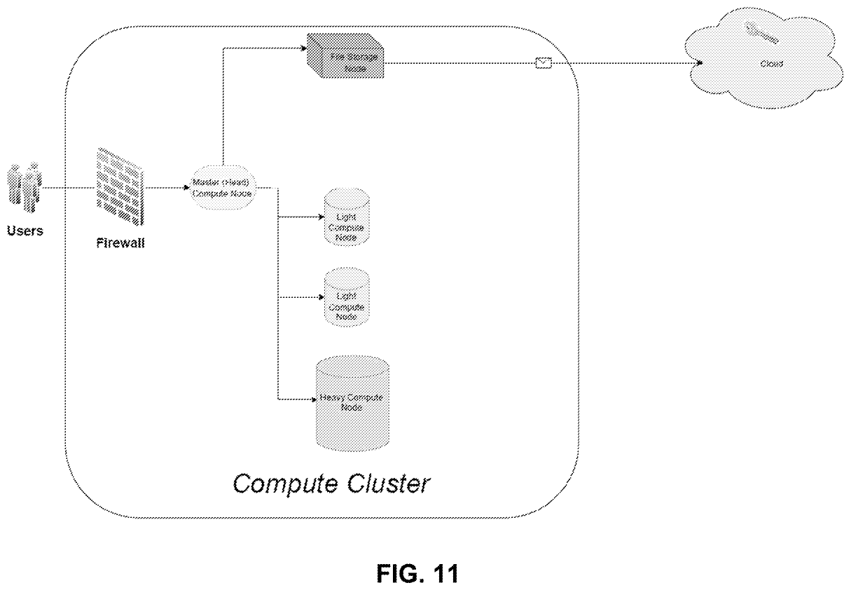

Referring to FIG. 11, a block diagram of a compute cluster is depicted, according to an embodiment. In one embodiment, there is a head node to deliver sample data and delegate jobs and tasks. The head node additionally stores sample data in a file storage node which can then deliver the sample data to a secure cloud service for backups. In an embodiment, a worker node with significant RAM can permit pre-loading of large scale genomic databases.

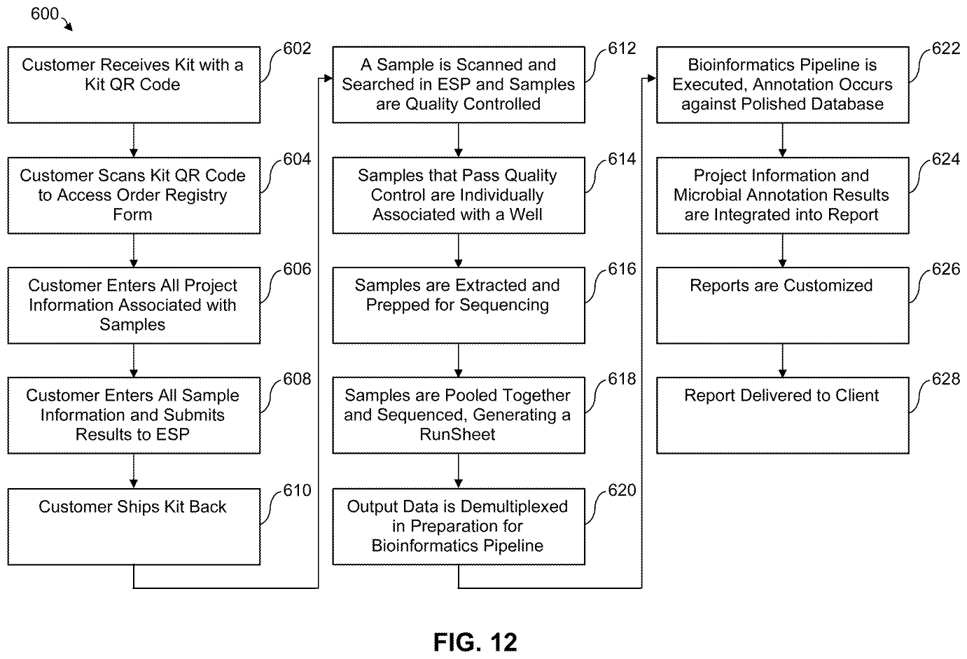

Referring to FIG. 12, a flow chart of a tracking overview process 600 is depicted. Tracking overview process 600 is a sample tracking use case for non-clinical samples, such as a surface test. At step 602, a user, often a customer, receives a surface test kit with a kit QR code and set number of 2D barcoded tubes. At step 604, the user scans the kit QR code to access the order and sample registry form. The order and sample registry form only allows users to enter a maximum number of tubes equal to the number of tubes received in the kit, simplifying the process for the user. At step 606, the user enters all project information which establishes all information associated with all samples included in the kit. In embodiments, project information can include the project name, project sampling date, the number of swabs in the project, and contact information for the user. The total number of swabs is set based on the QR code scanned previously.

At step 608, the user enters all sample information into the form. Sample information can include locational data such as a building and area description, a source identifier such as a countertop, a sample barcode which corresponds to a 2D barcode of a sample tube, and whether the sample is part of a cleaning proficiency test. Physical samples are associated with metadata via the user scanning in each respective barcode included within the kit. In one embodiment, the user must scan each barcode into the form using a mobile device, preventing the need of manual information entry. The form will additionally validate all barcodes. Upon completion of registering all samples in the kit, the user can click submit, which will transfer all submitted results in a consistent format to an ESP.

At step 610, the user is prompted to ship the kit back to the processing party. At this point, the sample barcodes and metadata are already electronically received by the processing party. In embodiments, the surface test kit can include a return label.

At step 612, once the surface test kit is received by the processing party, a sample is scanned and search in the ESP to bring up information for all samples included with the kit.

The samples are quality controlled for integrity. The samples which pass quality control are then moved to lab processing extraction.

At step 614, the samples are loaded onto an automated workstation that possesses a barcode scanner and associates each sample with the well position it is to be process in. The well position is then logged in the ESP for future reference and sequence file naming.

At step 616, Samples are extracted and prepped for sequencing, which includes each sample receiving a unique barcoded nucleic acid primer. The unique barcoded nucleic acid primer is used pose sequencing to demultiplex samples. Once extraction and library preparation is complete each individual sample includes a unique nucleic acid barcode tag for sequencing.

At step 618, all samples are pooled together and loaded onto a next-generation sequencing (NGS) machine, such as the Illumina NextSeq, for sequencing. A runsheet is generated within the ESP in which each respective sample included in the run is associated with its respective nucleic acid barcode, permitting seamless demultiplexing.

At step 620, upon completion of sequencing, the output data is demultiplexed in preparation for bioinformatics pipeline and deep annotation strategy analysis. Using the runsheet generated by the ESP, the user can initiate analysis prep with a click of a button. Analysis prep includes converting a large directory of basecalls into a directory of FASTQ sequence files, in which each file includes the original barcode in its name to make downstream metadata association possible.

At step 622, the bioinformatics pipeline and deep annotation strategy is executed, with the input directory of demultiplexed FASTQ files serving as input. Annotation occurs against a polished database and the end result is a text file consisting of total annotation counts for all microbes identified within each sample. The results are then ingested into the ESP for re-association with metadata and reporting.

At step 624, project information associated with the submitted surface test kit is pulled and integrated into the top section of the 2D report and microbial annotation results are integrated into the report for each sample/surface.

At step 626, reports are customized based on microbial annotation results and the user's desires. Taxa of interest can be further categorized with abundances tracked in each associated sample.

At step 628, the report is delivered to the client. In embodiments, the report can be delivered via encrypted email or an alternative delivery strategy.

A surface test kit can be used across a variety of industries, for example, the medical healthcare industry, the medical device industry, retain industry, construction industry, textile industry, food processing industry, food preparation industry, entertainment industry, sporting equipment industry, the sporting apparel industry, the lumber industry, schools and law enforcement, to name a few. A surface test kit can be used across a variety of locations in a particular industry, including medical facilities, nurse stations, breakrooms, schools, classrooms, recreational facilities, malls, public transportation, and gates/entrances. Samples can be collected from various surfaces or substrates, such as countertops, handles, ticket scanners, doorknobs, keyboards, medical devices, sporting equipment and containers.

A survey of an Intensive Care Unit showing a reduction of microbial diversity on surfaces post-intervention of a clean surfaces technology is included in the attached Appendix B, which is incorporated by reference in its entirety.

A paper titled Meta-omics Next-Generation Sequencing for Identification of Pathogens Causing Prosthetic Joint Infection is included in the attached Appendix C, which is incorporated by reference in its entirety.

A paper titled Meta-omics Next-Generation Sequencing for Identification of Pathogens Causing Prosthetic Joint Infection is included in the attached Appendix D, which is incorporated by reference in its entirety.

A project pitch detailing the commercialization and applications of a testing system to quickly and accurately identify root causes of infectious diseases is included in the attached Appendix E, which is incorporated by reference in its entirety.

A grant application detailing the benefits and applications of a bioinformatics pipeline and annotation systems for microbial genetic analysis is included in the attached appendix F, which is incorporated by reference in its entirety.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A computer-implemented method for rapidly identifying pathogen sequence data from raw sequence data generated by a sequencing system, comprising executing on a processor the steps of:
receiving, from the sequencing system, raw sequence data sequenced from a sample that includes both human and pathogen genetic material;
preprocessing the raw sequence data to filter out low quality sample sequence reads to generate a set of sample sequence reads;
extracting, via an alignment technique, pathogen sequence data from the set of sample sequence reads to create reporting results of matches for a subset of the sample sequence reads of pathogen genetic material, wherein the extracting includes:
comparing the set of sample sequence reads to host genomes representing known sequences of human genetic material stored in a host genome database to identify a human subset of the set of sample sequence that match a host genome sequence in the host genome database as background reads;
removing the background reads from the set of sample sequence reads to create a pathogen dataset of sample sequence reads that is stored separate from the set of sample sequence reads; and
comparing the pathogen dataset of sample sequence reads to a set of reference pathogen genomes representing known sequences of pathogen genetic material stored in a reference genome database to identify individual pathogens present in the pathogen dataset of sample sequence reads, including:
performing, via a k-mer annotation methodology, an initial fast annotation of the pathogen dataset to identify a subset of pathogens at a lower taxonomic rank (domain, phylum, class, order, family, genus); and
performing, via a sequence alignment methodology, a secondary slower annotation on the subset of pathogens classified at the lower taxonomic level to identify pathogens at a species level; and
storing the reporting results in a memory.

2. The computer-implemented method of claim 1 wherein the raw sequence data sequenced from the sample was bulk-filtered to enhance microbial RNA in the sample prior to sequencing by the sequencing system.

3. The computer-implemented method of claim 1 wherein the sequence alignment methodology is at least one of a local alignment process and a global alignment process.

4. The computer-implemented method of claim 1 further comprising:
preloading the host genome database into RAM prior to comparing, via k-mer annotation methodology, the set of sample sequence reads to host genomes.

5. The computer-implemented method of claim 1 further comprising:
preloading the reference genome database into RAM prior to comparing, k-mer annotation methodology, the pathogen dataset of sample sequence reads to a set of reference pathogen genomes.

6. The computer-implemented method of claim 1 wherein the reference genome database comprises a microbial polished database having an improved quality of pathogenic and nonpathogenic genomes within the microbial polished database that is achieved by selectively isolating regions of the genomes that are contaminated.

7. The computer-implemented method of claim 1, wherein the bioinformatics pipeline further comprises a deep annotation strategy that confers an additional task that can be scaled to a large number of taxa of interest each time a taxa of interest is extracted for re-annotation.

8. A bioinformatics pipeline designed to analyze next generation sequence data as input and systematically quality filter, normalize, annotate, quantify, and identify microbial taxa of interest contained within a microbial polished database implemented using the computer-implemented method of claim 1.

9. A bioinformatics pipeline implemented using the computer-implemented method of claim 1, wherein the bioinformatics pipeline includes a deep annotation strategy that confers an additional task that can be scaled to a large number of taxa of interest each time a taxa of interest is extracted for re-annotation.
